(19) United States
(12) Patent Application Publication
(10) Pub. No.: US 2006/0032507 A1
(43) Pub. Date: Feb. 16, 2006

(54) Contrast-enhanced ocular imaging

(76) Inventor: Hosheng Tu, Newport Coast, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/915,831

(22) Filed: Aug. 11, 2004

(51) Int. Cl. A61F 9/007

(52) U.S. Cl. 128/898; 623/4.1; 604/8

(57) ABSTRACT

The invention relates generally to medical devices and methods for ocular imaging and, more particularly, to devices and methods for increasing contrast in an eye in which an imaging contrast agent is introduced into an aqueous humor outflow channel. For example, in one embodiment, the outflow channel may be Schlemm's Canal, or in another embodiment, the outflow channel may be an episcleral vein. Also disclosed are methods for implanting a trabecular stent via an ab extemo procedure with assistance of enhanced magnetic resonance imaging to restore a part or all of the normal physiological function of directing aqueous outflow for maintaining a normal intraocular pressure in an eye.

### BACKGROUND OF THE INVENTION

1. Field of the Invention

The invention relates generally to medical devices and methods for ocular imaging and, more particularly, to devices and methods for increasing imaging contrast in an eye.

2. Description of the Related Art

The human eye is a specialized sensory organ capable of light reception and able to receive visual images. The trabecular meshwork serves as a drainage channel and is located in the anterior chamber angle formed between the iris and the cornea. The trabecular meshwork maintains a balanced pressure in the anterior chamber of the eye by draining aqueous humor from the anterior chamber.

About two percent of the United States population suffers from glaucoma. Glaucoma is a group of eye diseases encompassing a broad spectrum of clinical presentations, etiologies, and treatment modalities. Glaucoma causes pathological changes in the optic nerve, visible on the optic disk, and it causes corresponding visual field loss, resulting in blindness if untreated. Lowering intraocular pressure is the major treatment goal in all glaucomas.

In glaucomas associated with an elevation in eye pressure (intraocular hypertension), the source of resistance to outflow is mainly in the trabecular meshwork. The tissue of the trabecular meshwork allows the aqueous humor (“aqueous”) to enter Schlemm's canal, which then empties into aqueous collector channels in the posterior wall of Schlemm's canal and then into aqueous veins, which form the episcleral venous system. Aqueous is a transparent liquid that fills the region between the cornea, at the front of the eye, and the lens. Aqueous is continuously secreted by the ciliary body around the lens, so there is an essentially constant flow of aqueous from the ciliary body to the eye's anterior chamber. The eye's pressure is determined by a balance between the production of aqueous and its exit through the trabecular meshwork (major route) or uveal scleral outflow (minor route). The trabecular meshwork is located between the outer rim of the iris and back of the cornea, in the anterior chamber angle. The portion of the trabecular meshwork adjacent to Schlemm's canal (the juxtacanilicular meshwork) causes most of the resistance to aqueous outflow.

Glaucoma is grossly classified into two categories: closed-angle glaucoma, also known as angle closure glaucoma, and open-angle glaucoma. Closed-angle glaucoma is caused by closure of the anterior chamber angle by contact between the iris and the inner surface of the trabecular meshwork. Closure of this anatomical angle prevents normal drainage of aqueous from the anterior chamber of the eye.

Open-angle glaucoma is any glaucoma in which the angle of the anterior chamber remains open, but the exit of aqueous through the trabecular meshwork is diminished. The exact cause for diminished filtration is unknown for most cases of open-angle glaucoma. Primary open-angle glaucoma is the most common of the glaucomas, and it is often asymptomatic in the early to moderately advanced stage. Patients may suffer substantial, irreversible vision loss prior to diagnosis and treatment. However, there are secondary open-angle glaucomas which may include edema or swelling of the trabecular spaces (e.g., from corticosteroid use), abnormal pigment dispersion, or diseases such as hyperthyroidism that produce vascular congestion.

Current therapies for glaucoma are directed at decreasing intraocular pressure. Medical therapy includes topical ophthalmic drops or oral medications that reduce the production or increase the outflow of aqueous. However, these drug therapies for glaucoma are sometimes associated with significant side effects, such as headache, blurred vision, allergic reactions, death from cardiopulmonary complications, and potential interactions with other drugs. When drug therapy fails, surgical therapy is used. Surgical therapy for open-angle glaucoma consists of laser trabeculoplasty, trabeculectomy, and implantation of aqueous shunts after failure of trabeculectomy or if trabeculectomy is unlikely to succeed. Trabeculectomy is a major surgery that is widely used and is augmented with topically applied anticancer drugs, such as 5-flurouracil or mitomycin-C to decrease scarring and increase the likelihood of surgical success.

Approximately 100,000 trabeculectomies are performed on Medicare-age patients per year in the United States. This number would likely increase if the morbidity associated with trabeculectomy could be decreased. The current morbidity associated with trabeculectomy consists of failure (10-15%); infection (a life long risk of 2-5%); choroidal hemorrhage, a severe internal hemorrhage from low intraocular pressure, resulting in visual loss (1%); cataract formation; and hypotony maculopathy (potentially reversible visual loss from low intraocular pressure).

For these reasons, surgeons have tried for decades to develop a workable surgery for the trabecular meshwork.

The surgical techniques that have been tried and practiced are goniotomy/trabeculotomy and other mechanical disruptions of the trabecular meshwork, such as trabeculopuncture, goniophotoablation, laser trabecular ablation, and goniocurretage. These are all major operations and are briefly described below.

Goniotomy/Trabeculotomy: Goniotomy and trabeculectomy are simple and directed techniques of microsurgical dissection with mechanical disruption of the trabecular meshwork. These initially had early favorable responses in the treatment of open-angle glaucoma. However, long-term review of surgical results showed only limited success in adults. In retrospect, these procedures probably failed due to cellular repair and fibrosis mechanisms and a process of “filling in.” Filling in is a detrimental effect of collapsing and closing in of the created opening in the trabecular meshwork. Once the created openings close, the pressure builds back up and the surgery fails.

Trabeculopuncture: Q-switched Neodynium (Nd) YAG lasers also have been investigated as an optically invasive technique for creating full thickness holes in trabecular meshwork. However, the relatively small hole created by this trabeculopuncture technique exhibits a filling in effect and fails.

Goniophotoablation/Laser Trabecular Ablation: Goniophotoablation is disclosed by Berlin in U.S. Pat. No. 4,846,172 and involves the use of an excimer laser to treat glaucoma by ablating the trabecular meshwork. This was demonstrated not to succeed by clinical trial. Hill et al. used an Erbium:YAG laser to create full-thickness holes through trabecular meshwork (Hill et al., Lasers in Surgery and Medicine 11:341-346, 1991). This technique was investigated in a primate model and a limited human clinical trial at the University of California, Irvine. Although morbidity was zero in both trials, success rates did not warrant further human trials. Failure was again from filling in of surgically created defects in the trabecular meshwork by repair mechanisms. Neither of these is a viable surgical technique for the treatment of glaucoma.

Goniocurretage: This is an ab intemo (from the inside), mechanically disruptive technique that uses an instrument similar to a cyclodialysis spatula with a microcurrette at the tip. Initial results were similar to trabeculotomy: it failed due to repair mechanisms and a process of filling in.

Although trabeculectomy is the most commonly performed filtering surgery, viscocanulostomy (VC) and non-penetrating trabeculectomy (NPT) are two new variations of filtering surgery. These are ab extemo (from the outside), major ocular procedures in which Schlemm's canal is surgically exposed by making a large and very deep scleral flap. In the VC procedure, Schlemm's canal is cannulated and viscoelastic substance injected (which dilates Schlemm's canal and the aqueous collector channels). In the NPT procedure, the inner wall of Schlemm's canal is stripped of after surgically exposing the canal.

Trabeculectomy, VC, and NPT involve the formation of an opening or hole under the conjunctiva and scleral flap into the anterior chamber, such that aqueous is drained onto the surface of the eye or into the tissues located within the lateral wall of the eye. These surgical operations are major procedures with significant ocular morbidity. When trabeculectomy, VC, and NPT are thought to have a low chance for success, a number of implantable drainage devices have been used to ensure that the desired filtration and outflow of aqueous through the surgical opening will continue. The risk of placing a glaucoma drainage device also includes hemorrhage, infection, and diplopia (double vision).

Examples of implantable shunts and surgical methods for maintaining an opening for the release of aqueous from the anterior chamber of the eye to the sclera or space beneath the conjunctiva have been disclosed in, for example, U.S. Pat. No. 6,059,772 to Hsia et al., and U.S. Pat. No. 6,050,970 to Baerveldt.

All of the above surgeries and variations thereof have numerous disadvantages and moderate success rates. They involve substantial trauma to the eye and require great surgical skill in creating a hole through the full thickness of the sclera into the subconjunctival space. The procedures are generally performed in an operating room and have a prolonged recovery time for vision.

The complications of existing filtration surgery have prompted ophthalmic surgeons to find other approaches to lowering intraocular pressure or treating tissue of trabecular meshwork.

The trabecular meshwork and juxtacanilicular tissue provide the majority of resistance to the outflow of aqueous and, as such, are logical targets for tissue stimulation and/or rejuvenating in the treatment of open-angle glaucoma. In addition, minimal amounts of tissue are placed and functions of the existing physiological outflow pathways are restored.

As reported in Arch. Ophthalm. (2000) 118:412, glaucoma remains a leading cause of blindness, and filtration surgery remains an effective, important option in controlling the disease. However, existing filtering surgery techniques in any profound way to increase their effectiveness appears to have reached a dead end. The article further states that the time has come to search for new surgical approaches that may provide better and safer care for patients with glaucoma.

Therefore, there is a great clinical need for a method of treating glaucoma that is faster, safer, and less expensive than currently available drug or surgical modalities. The methods disclosed herein include ab extemo procedures that involve non-flap operations with assistance of imaging or visualization means for targeting the existing outflow pathways, including Schlemm's canal and collector ducts.

### SUMMARY OF THE INVENTION

Some embodiments disclosed herein relate to reliable visualization techniques, such as MRI or electromagnetic field imaging. In one embodiment, MRI-specific contrast agent is infused in a retrograde manner so as to fill at least a portion of the existing aqueous outflow pathway with MRI-specific contrast agent for enhanced MRI visualization in association with an ab extemo stent placement.

Further embodiments relate to a method of increasing contrast in an eye in which an imaging contrast agent is introduced into an aqueous humor outflow channel of the eye. For example, in one embodiment, the outflow channel may be Schlemm's Canal, or in another embodiment, the outflow channel may be an episcleral vein.

Some embodiments relate to a method for treating glaucoma of an eye comprising steps of providing a trabecular stent, wherein the stent comprises a first terminal and a second terminal having a lumen connecting the first and second terminals. Contrast agent may be infused retrogradely through an episcleral vein into an existing aqueous outflow pathway of the eye. The pathway may then be imaged using a contrast agent-enhanced imaging apparatus, and the stent may be injected ab externally toward the imaged pathway and advanced stent through the trabecular meshwork. The second terminal of the stent preferably is sized and shaped to be received within a portion of the existing aqueous outflow pathway, and the first terminal preferably is sized and shaped to be received within an anterior chamber of the eye. The stent preferably permits fluid communication from the first terminal in the anterior chamber to the second terminal in the pathway.

For purposes of summary, certain aspects, advantages and novel features have been described herein above. Of course, it is to be understood that not necessarily all such advantages may be achieved in accordance with any particular embodiment. Thus, the invention may be embodied or carried out in a manner that achieves or optimizes one advantage or group of advantages as taught or suggested herein without necessarily achieving other advantages as may be taught or suggested herein.

### BRIEF DESCRIPTION OF THE DRAWINGS

Having thus summarized the general nature of the invention and some of its features and advantages, certain preferred embodiments and modifications thereof will become apparent to those skilled in the art from the detailed description herein having reference to the figures that follow, of which:

FIG. 1 is a coronal cross-sectional view of an eye;

FIG. 2 is an enlarged cross-sectional view of an anterior chamber angle of the eye of FIG. 1;

FIG. 3 is a system for infusing contrast agent in a retrograde manner for enhancing magnetic resonance imaging on the outflow pathway; and

FIG. 4 is an ab externo stent implantation system guided with an enhanced magnetic resonance imaging.

### DETAILED DESCRIPTION OF THE PREFERRED EMBODIMENT

The preferred embodiments of the invention described herein relate particularly to surgical implantation of a trabecular stent for reduction of intraocular pressure ab externally with assistance of enhanced magnetic resonance imaging techniques. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described herein.

The trabecular meshwork and juxtacanilicular tissue together provide the majority of resistance to the outflow of aqueous and, as such, are logical targets for the treatment of glaucoma. Various embodiments of glaucoma devices and methods are disclosed herein for treating glaucoma by an ab externo procedure, with respect to trabecular meshwork. The “ab externo” procedure is herein intended to mean any procedure that creates an opening on the scleral wall through trabecular meshwork inwardly toward the anterior chamber. In most “ab externo” procedures disclosed herein, the direction of procedures passes through Schlemm's canal or a collector duct before entering the trabecular meshwork and approaching the anterior chamber. The trabecular meshwork can be generally said to be bordered on one side by the anterior chamber and on the other side by Schlemm's canal.

The “trabecular stent” is herein intended to mean a stent to support (that is, “to stent”) the trabecular meshwork when an opening or a slit is created inside the trabecular meshwork to allow fluid flowing through the trabecular meshwork. A trabecular stent is generally either hollow or porous for fluid transmission therethrough.

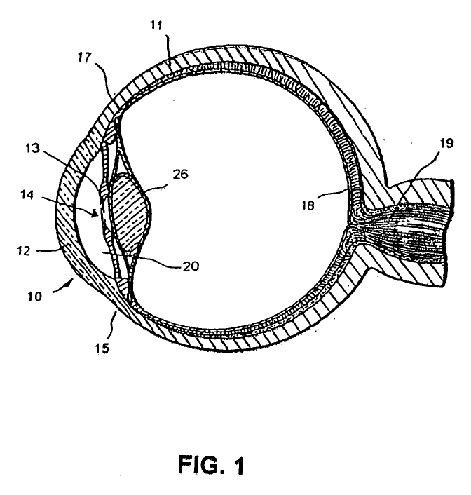

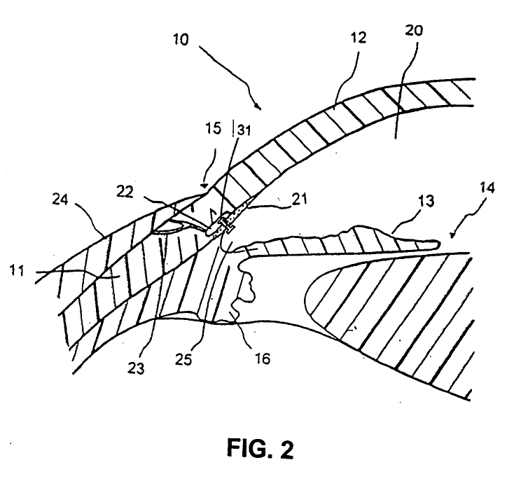

FIG. 1 is a cross-sectional view of an eye 10, while FIG. 2 is a close-up view showing the relative anatomical locations of a trabecular meshwork 21, an anterior chamber 20, and a Schlemm's canal 22. A sclera 11 is a thick collagenous tissue which covers the entire eye 10 except a portion which is covered by a cornea 12.

Referring to FIGS. 1 and 2, the cornea 12 is a thin transparent tissue that focuses and transmits light into the eye and through a pupil 14, which is a circular hole in the center of an iris 13 (colored portion of the eye). The cornea 12 merges into the sclera 11 at a juncture referred to as a limbus 15. A ciliary body 16 extends along the interior of the sclera 11 and is coextensive with a choroid 17. The choroid 17 is a vascular layer of the eye 10, located between the sclera 11 and a retina 18. An optic nerve 19 transmits visual information to the brain and is the anatomic structure that is progressively destroyed by glaucoma.

Still referring to FIGS. 1 and 2, the anterior chamber 20 of the eye 10, which is bound anteriorly by the cornea 12 and posteriorly by the iris 13 and a lens 26, is filled with aqueous humor (“aqueous”). Aqueous is produced primarily by the ciliary body 16, then moves anteriorly through the pupil 14 and reaches an anterior chamber angle 25, formed between the iris 13 and the cornea 12.

As best illustrated by the drawing of FIG. 2, in a normal eye, aqueous is removed from the anterior chamber 20 through the trabecular meshwork 21. Aqueous passes through the trabecular meshwork 21 into Schlemm's canal 22 and thereafter through a plurality of aqueous veins 23, which merge with blood-carrying veins, and into systemic venous circulation. Intraocular pressure is maintained by an intricate balance between secretion and outflow of aqueous in the manner described above. Glaucoma is, in most cases, characterized by an excessive buildup of aqueous in the anterior chamber 20 which leads to an increase in intraocular pressure. Fluids are relatively incompressible, and thus intraocular pressure is distributed relatively uniformly throughout the eye 10.

As shown in FIG. 2, the trabecular meshwork 21 is adjacent a small portion of the sclera 11. Exterior to the sclera 11 is a conjunctiva 24. Traditional procedures that create a hole or opening for implanting a device through the tissues of the conjunctiva 24 and sclera 11 involve extensive surgery, as compared to surgery for implanting a device, as described herein, which ultimately resides entirely within the confines of the sclera 11 and cornea 12.

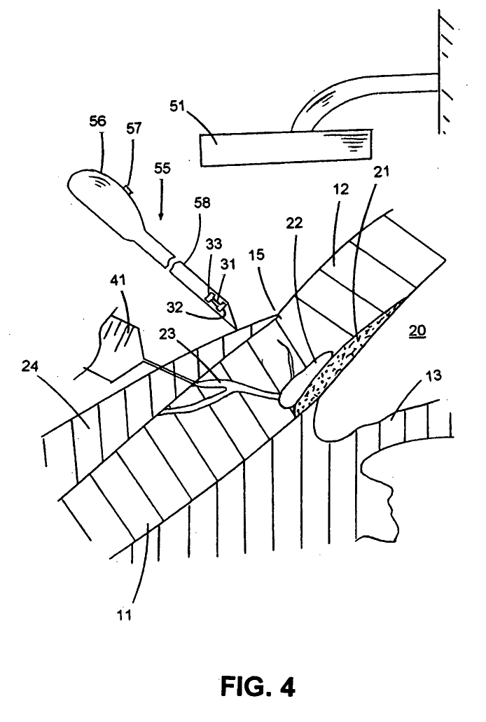

In accordance with one aspect of the invention, a trabecular stent 31 may be inserted ab externally, wherein the stent 31 comprises a first terminal 32 and a second terminal 33 having a lumen connecting the first and second terminals, as illustrated in FIG. 4. After implantation as shown in FIG. 2, the second terminal 33 of the stent 31 is sized and shaped to be received within a portion of the existing aqueous outflow pathway and the first terminal 32 is sized and shaped to be received within an anterior chamber 20 of the eye, and wherein the stent permits fluid communication from the first terminal in the anterior chamber to the second terminal in the pathway. In one embodiment, the existing aqueous outflow pathway may comprise Schlemm's canal 22, episcleral vein 23, intrascleral vein, and collector ducts that are spaced apart and generally connected to Schlemm's canal 22.

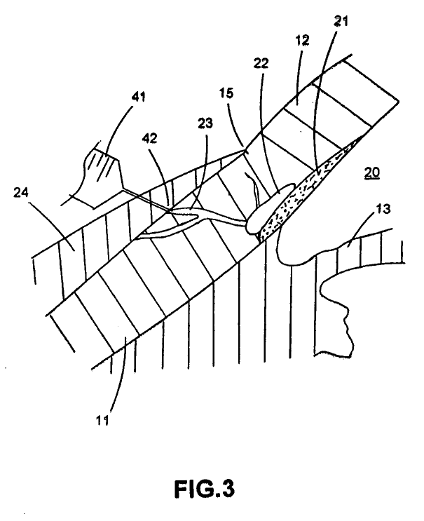

As shown in FIG. 3, an infusing system (for example, a syringe 41 or other fluid injector) may be used for infusing contrast agent retrogradely (in a retrograde manner) for enhancing contrast imaging (e.g., magnetic resonance) on the outflow pathway. In operation, the distal end 42 of the infusing system 41 is connected to one of the existing aqueous outflow pathways 23. A contrast agent (e.g., for an MRI) may be infused in a retrograde manner so as to fill at least a portion of the existing aqueous outflow pathway for enhanced visualization. Various contrast agents may be used for different visualization techniques. For example, iodinated contrast may be used for computed tomography (CT), ferromagnetic contrast for magnetic resonance, echogenic contrast for ultrasound, etc.

FIG. 4 shows an ab externo stent implantation system guided with an enhanced magnetic resonance imaging apparatus 51, wherein the stent implantation system 55 comprises a handpiece 56 and a push button 57 thereon for releasing a trabecular stent 31 out of the elongate injector 58. In one embodiment, the trabecular stent is an axisymmetric stent for easy loading onto and releasing from the elongate injector 58. Some aspects of the invention relate to a method for treating glaucoma of an eye comprising: (1) providing a trabecular stent, wherein the stent comprises a first terminal and a second terminal having a lumen connecting the first and second terminals; (2) infusing a contrast agent retrogradely through an episcleral vein into an existing aqueous outflow pathway of the eye; (3) imaging the pathway using a contrast agent-specific imaging apparatus; (4) injecting the stent ab externally toward the imaged pathway; and (5) advancing the stent through trabecular meshwork, wherein the second terminal of the stent is sized and shaped to be received within a portion of the existing aqueous outflow pathway and the first terminal is sized and shaped to be received within an anterior chamber of the eye, and wherein the stent permits fluid communication from the first terminal in the anterior chamber to the second terminal in the pathway.

The trabecular stent device of the exemplary embodiment may be manufactured or fabricated by a wide variety of techniques. These include, without limitation, by molding, extrusion, or other micro-machining techniques, among other suitable techniques.

The device 31 of the exemplary embodiment preferably comprises a biocompatible material such that inflammation arising due to irritation between the outer surface of the device and the surrounding tissue is minimized. Biocompatible materials which may be used for the device 31 preferably include, but are not limited to, titanium, titanium alloys, stainless steel, Nitinol, polypropylene, nylon, acrylic, PMMA (polymethyl methacrylate), medical grade silicone, e.g., Silastic™, available from Dow Corning Corporation of Midland, Mich.; and polyurethane, e.g., Pellethane™, also available from Dow Corning Corporation.

In other embodiments, the device of the embodiments may comprise other types of biocompatible material, such as, by way of example, polyvinyl alcohol, polyvinyl pyrolidone, collagen, heparinized collagen, polytetrafluoroethylene, expanded polytetrafluoroethylene, fluorinated polymer, fluorinated elastomer, flexible fused silica, polyolefin, polyester, polysilicon, and/or a mixture of the aforementioned biocompatible materials, and the like. In still other embodiments, composite biocompatible material may be used, wherein a surface material may be used in addition to one or more of the aforementioned materials. For example, such a surface material may include polytetrafluoroethylene (PTFE) (such as Teflon™), polyimide, hydrogel, heparin, therapeutic drugs (such as beta-adrenergic antagonists and other anti-glaucoma drugs, anti-scarring agents, or antibiotics), and the like.

In an exemplary embodiment of the trabecular meshwork surgery as shown in FIG. 4, the patient is placed in the supine position, prepped, draped and anesthetized as necessary. In one embodiment, a small (about less than 1 mm to a couple of millimeters) incision, which may be self-sealing, is made through the sclera 11 or cornea 12. The scleral incision can be made in a number of ways, for example, by using a self-trephining injector 58 or a micro-knife, among other tools.

Advantageously, the embodiments of the self-trephining injector 58 of the invention allow for a “one-step” procedure to make an incision in the sclera and to subsequently pierce through trabecular meshwork for stent implantation under the guidance of enhanced MRI. Desirably, this provides for a faster, safer, and less expensive surgical procedure as an ab extemo stent implantation allowing fluid communication from the first terminal 32 of the trabecular stent 31 in the anterior chamber to the second terminal 33 of the stent in one of the existing aqueous outflow pathways.

In the ab externo procedure of FIG. 4, the device is inserted into Schlemm's canal 22 with the aid of an injector or delivery apparatus that creates a small puncture into the eye 10 from sclera 11. An imaging technique, such as MRI or electromagnetic field imaging technique, is utilized. The imaging provides guidance for the insertion of the device.

Some aspects of the invention relate to a medical device system for treating glaucoma that uses OCT (optical coherence tomography) as an imaging and locating system for trabecular stent placement. In one embodiment, the procedure would first be set up with triangulation or some means to reliably establish the implant location in x, y, and z coordinates by using OCT within a few microns, most preferably in a non-invasively and non-contact manner. Having acquired the target space or location, the trabecular stent would then be injected into place via an ab externo procedure. In one embodiment, OCT-specific contrast agent is infused in a retrograde manner so as to fill at least a portion of the existing aqueous outflow pathway with OCT-specific contrast agent for enhanced OCT visualization.

In an ab extemo procedure for implanting a trabecular stent through trabecular meshwork, an imaging system of MRI type is provided to guide the placement of the implant. More particularly, the surgery is to place a trabecular stent under MRI guidance, wherein the stent has one end of the stent exposed to the anterior chamber and the other end to Schlemm's canal for fluid communication therebetween. Some aspects of the invention relating to infusing a MRI-specific contrast agent retrogradely through an episcleral vein into an existing aqueous outflow pathway of the eye.

It is one aspect of the present invention to provide an imaging technique which could be used as an enabling technology to perform a precision, guided, ab-externo or ab-interno implantation of a device or substance into a specific location in Schlemm's canal, the trabecular meshwork, or the collector duct system to treat or prevent glaucoma or its symptoms. The device could be placed through, or around the trabecular meshwork, bypassing the diseased area that resists normal flow of aqueous.

Some of the imaging techniques that may have practical applicability in the glaucoma stent placement include MRI, electromagnetic imaging, Tetrahertz imaging, computerized tomography, phase contrast imaging, X-ray (or fluorescent imaging), optical, ultrasound, and the like. Some aspects of the invention relate to the reliable visualization technique including MRI, electromagnetic field, and focused sonic/ultrasonic/acoustic technique that is equivalent to a focused light technique known as “laser.” In one aspect of the present disclosure, the focused sonic, ultrasonic or acoustic technique is to have collimated sound, ultrasound or acoustic waves for visualization purposes.

One imaging modality, which has the potential to supplant fluoroscopy, or perhaps replace it in the long term, is magnetic resonance imaging (MRI). MRI does not use ionizing radiation and does not require catheterization to image vasculature. MRI contrast agents, which are often necessary for best resolution, are much less nephrotoxic than iodinated fluoroscopy agents and are effective when administered intravenously through episcleral veins.

One advantage of MRI is that different scanning planes and slice thicknesses can be selected without loss of resolution. This selection permits high quality transverse, coronal and sagittal images to be obtained directly. MRI has excellent soft tissue contrast and tissue discrimination because there are at least four separate variables that can determine MRI signal intensity: (1) spin-lattice (longitudinal) relaxation time, (ii) spin-spin (transverse) relaxation time, (iii) proton density, and (iv) flow. MRI is presently used for diagnostic applications, but interventional magnetic resonance (iMR) angiography is an active area of research. For example, MRI guided balloon angioplasty has been performed to demonstrate feasibility. MRI guided arrhythmia mapping has been recently performed in catheter-based tissue ablation. Similarly, cardiovascular stent placement in humans under MRI has also been demonstrated. Some aspect of the invention is to provide a trabecular stent placement ab externally under MRI or enhanced MRI.

Magnetic resonance imaging (MRI) is a sophisticated diagnostic technique that uses a magnetic field, radiowaves and a computer to generate detailed, cross-sectional images of human anatomy. Because it produces excellent soft-tissue images, MRI is most commonly used to image the brain, spine, thorax, vascular system and musculoskeletal system (including the knee and shoulder).

During an MRI exam, the patient is placed inside a scanner that produces a static magnetic field up to 8,000 times stronger than the earth's own magnetic field. Exposure to this force causes the hydrogen protons within the patient's body to align with the magnetic field. When a radiofrequency pulse is applied, the protons spin perpendicular to the magnetic field. As the protons relax back into alignment with the magnetic field, a signal is sent to a radiofrequency coil that acts as an antenna. This signal then is processed by a computer. Different tissues produce different signals. For example, protons in water relax more slowly than those in fat. This differentiation can be detected, measured and converted into a cross-sectional image of the patient's anatomy. Unlike other scans, MRI images can be taken from almost any angle without moving the person around.

The technique of MRI encompasses the detection of certain atomic nuclei (those possessing magnetic dipole moments) utilizing magnetic fields and radiofrequency radiation. It is similar in some respects to x-ray computed tomography in providing a cross-sectional display of the body organ anatomy, only with excellent resolution of soft tissue detail. In its current use, the images constitute a distribution map of protons, and their properties, in organs and tissues. The fundamental lack of any known hazard associated with the level of the magnetic and radiofrequency fields that are employed renders it possible to make repeated scans on vulnerable individuals. Additionally, any scan plane can readily be selected, including transverse, coronal, and sagittal sections. MRI is, therefore, a safe non-invasive technique for medical imaging to assist stent implantation.

The hydrogen atom, having a nucleus consisting of a single unpaired proton, has one of the strongest magnetic dipole moments of nuclei found in biological tissues. Since hydrogen occurs in both water and lipids, it is abundant in the human body. Therefore, MRI is most commonly used to produce images based upon the distribution density of protons and/or the relaxation times of protons in organs and tissues. Other nuclei having a net magnetic dipole moment also exhibit a nuclear magnetic resonance phenomenon which may be used in MRI applications. Such nuclei include carbon-13 (six protons and seven neutrons), fluorine-19 (9 protons and 10 neutrons), sodium-23 (11 protons and 12 neutrons), and phosphorus-31 (15 protons and 16 neutrons).

For most types of exams, the MR technologist will wrap a special coil around the eye that is being examined. This coil helps concentrate the radiofrequency pulses. The MR technologist then will position the patient on a padded, movable table that will slide into the opening of the scanner. A contrast agent to highlight internal organs and structures is given through an episcleral vein retrogradely. The contrast changes the relaxation rate of protons in the body, illuminating organs and tissues and making the existing aqueous outflow pathway appear brighter for stent placement ab externally.

In order to achieve effective contrast between MR images of different tissue types, MR contrast agents (e.g. paramagnetic metal species) may be administered to the subject, which affect relaxation times in the zones in which they are administered or at which they congregate. By shortening the relaxation times of the imaging nuclei (the nuclei whose MR signal is used to generate the image) the strength of the MR signal is changed and image contrast is enhanced.

“Diagnostic agent” refers herein to any agent which may be used in connection with methods for imaging an internal region of a patient and/or diagnosing the presence or absence of a disease in a patient. Exemplary diagnostic agents include, for example, contrast agents for use in connection with ultrasound imaging, magnetic resonance imaging or computed tomography imaging of a patient. Diagnostic agents may also include any other agents useful in facilitating diagnosis of a disease or other condition in a patient, whether or not imaging methodology is employed.

Nitroxides are paramagnetic contrast agents which increase both spin-lattice relaxation and spin-spin relaxation rates on MRI by virtue of the presence of an unpaired electron in the nitroxide molecule. The paramagnetic effectiveness of a given compound, such as an MRI contrast agent, may be related, at least in part, to the number of unpaired electrons in the paramagnetic nucleus or molecule, and specifically, to the square of the number of unpaired electrons. For example, gadolinium has seven unpaired electrons whereas a nitroxide molecule has one unpaired electron. Thus, gadolinium is generally a much stronger MRI contrast agent than nitroxide. However, effective correlation time, another important parameter for assessing the effectiveness of contrast agents, confers potential increased relaxivity to the nitroxides. When the tumbling rate is slowed, for example, by attaching the paramagnetic contrast agent to a large molecule, it will tumble more slowly and thereby more effectively transfer energy to hasten relaxation of the water protons. In gadolinium, however, the electron spin relaxation time is rapid and will limit the extent to which slow rotational correlation times can increase relaxivity. For nitroxides, however, the electron spin correlation times are more favorable and tremendous increases in relaxivity may be attained by slowing the rotational correlation time of these molecules.

The gas filled vesicles are ideal for attaining the goals of slowed rotational correlation times and resultant improvement in MRI relaxivity. The vesicles are generally in sub-micron or nanometer size ranges. Although not intending to be bound by any particular theory of operation, it is contemplated that since the nitroxides may be designed to coat the perimeters of the vesicles, for example, by making alkyl derivatives thereof, the resulting correlation times can be optimized. Moreover, the resulting contrast medium of the present invention may be viewed as a magnetic sphere, a geometric configuration which maximizes relaxivity.

Exemplary superparamagnetic contrast agents suitable for use in the compositions of the present invention include metal oxides and sulfides which experience a magnetic domain, ferro- or ferrimagnetic compounds, such as pure iron, magnetic iron oxide, such as magnetite, γ-Fe₂O₃, Fe₃O₄, manganese ferrite, cobalt ferrite and nickel ferrite. Paramagnetic gases can also be employed in the present compositions, such as oxygen 17 gas (¹⁷O₂). In addition, hyperpolarized xenon, neon, or helium gas may also be employed. MR whole body imaging may then be employed to rapidly screen the body, for example, for thrombosis.

The contrast agents, such as the paramagnetic and superparamagnetic contrast agents described above, may be employed as a component within the lipid and/or vesicle compositions. In the case of vesicle compositions, the aforementioned contrast agents may be entrapped within the internal void thereof, administered as a solution with the vesicles, incorporated with any additional stabilizing materials, or coated onto the surface or membrane of the vesicle. Mixtures of any one or more of the paramagnetic agents and/or superparamagnetic agents in the present compositions may be used. The paramagnetic and superparamagnetic agents may also be co-administered separately, if desired. Iron-containing MRI (magnetic resonance imaging) contrast agents (also called superparamagnetic agents) are used to help provide a clear picture during MRI. MRI is a special kind of diagnostic procedure. It uses magnets and computers to create images or “pictures” of certain areas inside the body.

Ferumoxides, an iron-containing contrast agent, is given by injection before MRI to help find and diagnose tumors of the liver. The dose of ferumoxides will be different for different patients according to body weight. Ferumoxides are to be used only by or under the supervision of a doctor. In one aspect, ferumoxides are used retrogradely through an episcleral vein for stent placement under enhanced MRI.

If desired, the paramagnetic or superparamagnetic agents may be delivered as alkylated or other derivatives incorporated into the compositions, especially the lipidic walls of the vesicles. The ideal situation in terms of maximizing the contrast effect would be to make the iron oxide particles hollow, flexible and as large as possible. It has not been possible to achieve this heretofore and it is believed that the benefits have been unrecognized heretofore also. By coating the inner or outer surfaces of the vesicles with the contrast agents, even though the individual contrast agents, for example, iron oxide nanoparticles or paramagnetic ions, are relatively small structures, the effectiveness of the contrast agents may be greatly enhanced. In so doing, the contrast agents may function as an effectively much larger sphere wherein the effective domain of magnetization is determined by the diameter of the vesicle and is maximal at the surface of the vesicle. These agents afford the advantage of flexibility, namely, compliance. These flexible vesicles slide through the capillaries much more easily.

For optical imaging, optically active gases, such as argon or neon, may be incorporated in the present compositions of contrast agents. In addition, optically active materials, for example, fluorescent materials, including porphyrin derivatives, may also be used as contrast agents.

Novel targeted therapeutic delivery systems of the present invention are useful as contrast media in diagnostic imaging, and for use in all areas where diagnostic imaging is employed. Diagnostic imaging is a means to visualize internal body regions of a patient, and includes, for example, ultrasound (US), magnetic resonance imaging (MRI), nuclear magnetic resonance (NMR), computed tomography (CT), electron spin resonance (ESR); nuclear medicine when the contrast medium includes radioactive material; and optical imaging, particularly with a fluorescent contrast medium. Diagnostic imaging also includes promoting the rupture of vesicles via the methods of the present invention. For example, ultrasound may be used to visualize the vesicles and verify the localization of the vesicles in certain tissue. In addition, ultrasound may be used to promote rupture of the vesicles once the vesicles reach the intended target, including tissue and/or receptor destinations, thus releasing a bioactive agent, such as anti-glaucoma drugs. Similarly, light or laser may be used to visualize the vesicles and verify the localization of the vesicles in certain tissue. In addition, light or laser may be used to promote rupture of the vesicles once the vesicles reach the intended target, including tissue and/or receptor destinations, thus releasing a bioactive agent, such as anti-glaucoma drugs.

Nuclear Magnetic Resonance (NMR) is a non-invasive technique using radiofrequency waves to investigate the nuclei of atoms in a magnetic field. NMR spectroscopy primarily provides information regarding chemical structures while MRI provides spatial information. Since the techniques are non-invasive, any given outflow pathway can serve as its own control and reveal diagnostic changes with respect to an intervention or therapy.

A contrast effective amount of the diagnostic agent containing composition is that amount necessary to provide tissue visualization with, for example, magnetic resonance imaging or x-ray imaging. Means for determining a contrast effective amount in a particular subject will depend, as is well known in the art, on the nature of the magnetically reactive material used, the mass of the subject being imaged, the sensitivity of the magnetic resonance or x-ray imaging system and the like.

While the components and techniques of the invention have been described with a certain degree of particularity, it is manifest that many changes may be made in the specific designs, constructions and methodology herein above described without departing from the spirit and scope of this disclosure. It should be understood that the invention is not limited to the embodiments set forth herein for purposes of exemplification, but is to be defined only by a fair reading of the appended claims, including the full range of equivalency to which each element thereof is entitled.

What is claimed is:

1. A method of increasing contrast in an eye, the method comprising:
introducing an imaging contrast agent into an aqueous humor outflow channel of the eye.

2. The method of claim 1, wherein the outflow channel is Schlemm's Canal.

3. The method of claim 1, wherein the outflow channel is an aqueous collector channel.

4. The method of claim 1, wherein the outflow channel is an episcleral vein.

5. The method of claim 1, wherein the contrast agent is an iodinated contrast agent.

6. The method of claim 1, wherein the contrast agent is a ferromagnetic contrast agent.

7. The method of claim 1, wherein the contrast agent is an echogenic contrast agent.

8. The method of claim 7, wherein the echogenic contrast agent comprises microbubbles.

9. The method of claim 1, wherein said introducing comprises injecting.

10. The method of claim 5, further comprising imaging said eye with Computed X-ray Tomography.

11. The method of claim 6, further comprising imaging said eye with magnetic resonance.

12. The method of claim 7, further comprising imaging said eye with ultrasound.

13. The method of claim 1, further comprising inserting an implant in the eye such that an inlet portion of the implant conducts fluid from the anterior chamber to an outlet portion of the implant residing outside the anterior chamber.
